(19) United States
(12) Patent Application Publication
(10) Pub. No.: US 2022/0369960 A1
(43) Pub. Date: Nov. 24, 2022

(54) PROCESS FOR MANAGING OR ASSISTING IN THE MONITORING OF A PHYSIOLOGICAL PARAMETER OF AN INDIVIDUAL, IN PARTICULAR THE BLOOD SUGAR LEVEL

(71) Applicant: DOCTEGESTIO, PARIS (FR)

(72) Inventor: Benoît MIRAMBEAU, MOIRAX (FR)

(21) Appl. No.: 17/285,400

(22) PCT Filed: Jun. 28, 2019
(86) PCT No.: PCT/EP2019/067303

§ 371 Date: Aug. 11, 2022

(30) Foreign Application Priority Data
Jun. 28, 2018 (FR) 1870770

(51) Int. Cl. A61B 5/145

(52) U.S. Cl. CPC A61B 5/14532; G16H 20/60

(57) ABSTRACT

A method for assisting in the control of a blood glucose-type physiological parameter of an individual, including the steps of prior real-time recording in the database of information relating to an insulin dose adaptation protocol, this protocol being specific to the individual, collecting a value for the physiological parameter at a given time and recording the value for the parameter in a database, recording in the database of values collected for the physiological parameter at different moments in time, optionally, recording in the database of data items relating to the individual's nutrition and/or physical activity, geolocation of the individual, and processing of information to automatically calculate at least one insulin dose.

### CROSS-REFERENCES TO RELATED APPLICATIONS

This application claims the benefit of the International Application No. PCT/EP2019/067303, filed on Jun. 28, 2019, and of the French patent application No. 1870770 filed on Jun. 28, 2018, the entire disclosures of which are incorporated herein by way of reference.

### FIELD OF THE INVENTION

The invention relates to a method for managing or assisting in the control, particularly the regulation, of a physiological parameter of an individual, and for automated calculation of a quantity that is dependent on this physiological parameter and on other parameters.

### BACKGROUND OF THE INVENTION

The invention will be described more particularly with regard to a chronic pathology such as diabetes, but it is not limited thereto. It can be applied to other pathologies such as hypertension or hemophilia, or to other types of assistance or monitoring associated with a physiological parameter that requires regular monitoring.

People with diabetes need to check their blood glucose several times throughout the day in an attempt to optimize the insulin doses they need to administer to themselves.

Each diabetic has their own protocol for administering doses of insulin that has been developed by a specialist practitioner such as a diabetologist. A protocol depends particularly on the patient's age, sex, eating habits, and the history of blood glucose levels measured, particularly at least from the previous day, or even from the last few days in the same time slot. This protocol is presented as a “roadmap” based on:
different time slots over a day, for example breakfast, morning, lunch, afternoon, dinner, evening, and bedtime;
a minimum threshold value for the blood glucose level and a maximum threshold value; and
a proportion of increase or decrease in a number of insulin dose units according to the history of blood glucose levels for the time slots.

Therefore, not only does the patient need to measure their blood glucose level periodically, but they must also calculate the dose of insulin to be injected every day and several times a day, which may be either the same as the previous day for same time slot or different, representing a decrease or an increase in the dose. It is therefore necessary to have the level from the previous day for each time slot and to know how to calculate the right dosage of insulin by adding or subtracting dose units.

However, the calculation can be complex and tedious to carry out, particularly for children, and it is very restrictive in any case, with a non-negligible risk of error.

In addition, the planned increases and decreases in doses are generally fixed when the individual has not performed any unusual exertion since the previous day. However, when the individual has performed a physical activity, for example, the protocol is no longer suitable, and the individual manages their insulin doses as best they can according to their momentary blood glucose level until the day after the exertion upon waking up.

Software applications now exist for helping the individual control their blood glucose level and facilitate the calculation of daily insulin doses. Some applications also take carbohydrate intakes from meals into account in order to help calculate the dose of insulin that seems most suitable.

However, it turns out that physical activity and even other parameters can play a role in the variation of the blood glucose level. Today, diabetics do not have any automated means to help them control their blood sugar and calculate insulin doses based not only on the measurement of the blood sugar level and the carbohydrate level associated with their current nutrition, but also other influencing parameters.

In addition, protocols can be very different for different individuals. And yet the existing software applications for calculating the insulin dose have standard protocols which are already integrated, each associated with a calculation algorithm that is fixed in the application. It is thus impossible to take individualized protocols into account.

### SUMMARY OF THE INVENTION

It is therefore an object of the invention to provide a method for managing or helping to control a physiological parameter, particularly associated with a chronic pathology such as diabetes, which can be adapted to any management/control protocol for the parameter, and can take various parameters into account that are external to the individual in order to calculate a quantity influencing the physiological parameter as accurately as possible and even to provide a forecast of the results.

According to the invention, the method for managing or assisting in the control or regulation of a physiological parameter of an individual comprises the steps of:

collecting a value for the physiological parameter at a given time and recording the value for the parameter in a database,

recording in the database of values collected from the physiological parameter at different moments in time (in order to constitute a history of values associated with a precise moment or time slot in the database),

optionally, recording in the database of data items relating to the individual's nutrition and/or physical activity,

automated calculation of at least one quantity influencing the physiological parameter,

the method further comprising a step of recording (in real time by the individual themself) in the database of information relating to an individual-specific control protocol such as adaptation of the quantity influencing the physiological parameter, and a step of processing the information, using electronic processing means and a computer program, in order to transform it (in real time) into a mathematical calculation algorithm capable of handling the automated calculation of the quantity influencing the physiological parameter (each time the calculation is implemented, particularly immediately after the step of collecting a value for the physiological parameter at a given time and recording it in the database).

The step of entering protocol information in order to transform it into a calculation algorithm is done in real time by means of a dedicated software application, meaning that it is done by the user or an authorized third party via the software application after the software application is made available to the user. The entry of information is carried out in real time (generally only once after loading the software application and before its use in the collection of the data for the physiological parameter, and with the possibility of modifying the information later), meaning that the related information in the protocol is in no way pre-recorded in the program (algorithm) for calculating the quantity influencing the physiological parameter. The invention therefore allows each individual to enter their own personalized management or control protocol in order to obtain a fully personalized calculation, and not to provide a calculation from a basic protocol that is already included in the calculation method (already included in the computer calculation program and not modifiable by the user). The user/authorized third party therefore has control over the entering of information relating to the protocol, and it can be modified at any time.

According to one feature,

the physiological parameter to be monitored is the blood glucose level, the quantity influencing the physiological parameter is an insulin dose, and the monitoring protocol is the protocol for adapting insulin doses; or

the physiological parameter to be controlled is blood pressure, and the control protocol is the protocol for adapting a quantity influencing blood pressure in the event of hypertension, or

the physiological parameter to be monitored is the prothrombin level and the control protocol is the protocol for adapting a quantity influencing the prothrombin level in the event of hemophilia.

In a particular embodiment, the method for managing or assisting in the control or regulation of a physiological parameter of an individual comprises managing or assisting in the control or regulation of the blood glucose level of an individual, and the method comprises the following steps:
a preliminary step preceding at least the first implementation of the method, which comprises recording (in real time) in a database information relating to an individual-specific protocol for adapting insulin doses, the recording being implemented by at least one dedicated software application and an entry and display interface system of the computer, tablet, or smartphone type;
collecting the blood glucose level at a given time and recording the value for the blood glucose level in the database, the collection of the value for the blood glucose level being carried out by measuring it, and by recording it manually or orally, or even in an automated manner via a connected measurement system, on an entry and display interface system (which is not necessarily the same as the preliminary step but which constitutes the system used on a daily basis by the individual to manage their blood glucose);
recording in the database of values collected from the blood glucose level at different moments in time in order to constitute a history of values of the individual's blood glucose level, each of the values being associated with a precise moment, such as a precise time slot;
optionally, recording (for example recording in the preliminary step of entering the protocol, or occasionally during an implementation of the method in order to calculate a dose) in the database via an interface system for entering and displaying data relating to the individual's nutrition and/or physical activity;
recording in the database of the individual's location, which is preferably recorded automatically by the geolocation of the entry and display interface system used during the collection step;
processing of the recorded information using electronic processing means and a computer program to calculate (in real time) in an automated manner at least one dose of insulin from the information of the individual-specific protocol, historical data on blood glucose levels, location, and optionally data relating to nutrition and/or physical activity.

According to the method of the invention, after the step of recording in the database of information relating to the individual-specific protocol for adapting insulin doses, and before the regular implementation of the management method by the individual, the method comprises a step of processing the information of the protocol, using electronic processing means and a computer program, in order to transform them (in real time) into a mathematical calculation algorithm which will subsequently provide, also on the basis of other data that will be recorded at least during the collection of a blood glucose level and the geolocation, for the automated calculation of the insulin doses at a given time for the individual.

The step of automated calculation by means of the dedicated software application (based on the calculation algorithm, which is developed in real time) to calculate at least one insulin dose that is suitable for the present moment and impacts the blood glucose level is thus carried on the basis of at least the prerecorded, individual-specific adaptation protocol and the individual's historical blood glucose level values, as well as nutrition-related data if these have just been recorded because they were required for real-time calculation of the dose to be administered, as well as the data pertaining to geolocation that can influence the dose to be administered (particularly as a function of the atmospheric pressure and/or the temperature of the place where the individual is located).

The method compares, in particular, one or more blood sugar levels collected previously and, if necessary, calculates decreases or increases in the insulin dose according to the recorded personalized adaptation protocol.

The step of recording information in the database relating to the individual-specific adaptation protocol concerns information relating to the personalized protocol set up for the individual for the regulation of their blood glucose level, this adaptation protocol having been put in place by the individual's practitioner; this step is necessarily carried out before the first use/implementation of the method by the individual in order to then regularly calculate their insulin doses. Personalized protocol information is entered by the user or a third party. The dedicated software application is designed precisely for entering the personalized protocol and for modifying the data of the personalized protocol at a later time (in the event of a modification by the practitioner of the individual's adaptation protocol). The adaptation protocol comprises indicating to the individual how to adapt their insulin doses according to the time of day, taking into account the blood glucose levels from the previous day and that day.

The method of the invention turns out to be completely innovative with respect to the prior art. Until now, the assistance or control methods and their software applications made it possible, with the real-time data entry of information relating to a meal that the individual is about to have or to the physical exercise that they are going to perform, to indicate a recommended insulin dose, this dose calculation being based on a dose calculation algorithm that is already included in the software application when the software application is made available to an individual. On the one hand, this calculation algorithm of the prior art is generic in order to apply to a larger number of individuals (at least in terms of sex and weight). On the other hand, this generic calculation algorithm cannot be modified in real time.

However, unlike the prior art, the method of the invention is completely personalized; in a preliminary step, it involves entering all of the information from the insulin dose administration/adaptation protocol that was prescribed in a personal capacity by the practitioner for an individual who is to use the method. In addition, it has the advantage, once the preliminary step has been carried out and the calculation algorithm has been generated in real time, of taking the individual's historical blood glucose levels as well as their geolocation into account during its implementation. In addition, it will always be possible over time, if the practitioner changes the dose administration protocol, to modify, by means of the dedicated software application, the protocol that was recorded initially or previously in order to generate a new algorithm for calculating the doses.

According to the invention, the method for controlling the blood glucose level comprises different steps associated with various parameters that are associated not only with the measurement as such of the blood glucose level—these parameters being associated particularly with the individual's nutrition and/or physical activity and the individualized protocol for adapting insulin doses—but also with the individual-specific history of blood glucose levels.

The method makes it possible, based on the history of blood glucose levels and the insulin administration protocol established by the doctor specifically for the individual, to calculate the ad hoc insulin dose that the patient needs to administer to themself automatically and in real time.

The step of entering information for the recording of the protocol is preferably recorded by the user's doctor (authorized third party). Thus, there is no longer any risk of a misunderstanding of the adaptation protocol, since it is preferably entered by the practitioner, and, above all, the individual is no longer at risk of making a mistake in calculating the adjustment of the doses and thus causing overdoses or under-dosages of insulin.

The control method of the invention also makes it possible to record in a database the historical blood glucose levels combined with nutrition and physical activity in order to deduce therefrom statistical data that are to be subsequently processed by practitioners, for example, in order to develop treatment protocols or carry out studies on changes in blood glucose levels according to the type of individual, the type of physical activity, and the type of nutrition.

According to one particular feature, the information in the protocol comprises data relating to the individual's physical activity.

According to yet another feature, the step of recording in the database information relating to the individual's protocol is carried out only once (by the user or an authorized third party) before the regular implementation of the method by the user, it being possible for the protocol to be updated by recording new associated information (i.e., the protocol information can be changed at any time). The recording of the protocol comprises the manual entry of the doctor's handwritten prescription via a software application on an entry and display interface system, the computer program translating the entry into the algorithm for calculating the quantity influencing the physiological parameter, e.g., insulin doses.

When the method is applied to diabetes, the automated calculation of an insulin dose at a given time is a function of at least one previous value for the blood glucose level, a low threshold value for the blood glucose level, and/or a high threshold value for the blood glucose level, a weighting coefficient based on data relating to physical activity, and optionally a carbohydrate level based on nutrition-related data.

Advantageously, the method comprises a step of recording and processing additional data during the collection of a value for the quantity influencing the physiological parameter (such as the collection of the blood glucose level), these additional data being the individual's location, the weather, and/or the temperature and/or altitude and/or altitude and/or atmospheric pressure and/or humidity, and/or the individual's state of mind.

The collection of the value for the quantity influencing the physiological parameter is carried out by measuring the quantity (such as the blood glucose level or the blood pressure or the prothrombin level) and by recording it manually or orally, or even in an automated manner by means of a connected measurement system, on an interface system between the individual and the database.

Preferably, the method further comprises at least one step of alert notification to the user/individual and/or the sending of alerts to third parties, automatically and/or voluntarily by the individual as a function of the result of the value for the quantity influencing the physiological parameter (e.g., as a function of the result of the blood glucose level, particularly in the event of hypo- or hyperglycemia), and/or a lack of implementation of the control method, and/or when the quantity (such as the insulin dose) influencing the physiological parameter and recorded during an additional recording step does not correspond to the quantity (such as the insulin dose) calculated.

The protocol Information to be recorded is entered on an entry and display interface system through step-by-step display and entry into data fields. For diabetes, the information in the insulin dose adjustment protocol is:

the type of diabetes;

the type of device used to administer the doses (pen or pump);

the unit of insulin dose used (mmol/L or mg/dL or g/L);

low and high threshold values for blood glucose levels;

the control periods, such as night, breakfast, morning, lunch, afternoon, dinner, evening, with a start time and an end time being assigned to each period;

meals, when a meal is associated with a control period;

insulin-related information for each period, particularly the type of insulin, the name of the insulin, whether it is a functional insulin therapy, and the range of insulin dose according to a level value for blood glucose (several ranges can be entered that are associated with a blood glucose level value);

the possible adjustment of the insulin dose for a given period;

in the event of an adaptation, the selection of the adaptation method from among the methods offered on the entry and display interface system, particularly from among the known methods used by practitioners and called “the traditional method,” “the method by difference,” and “the difference method Δ”;

in the event of an adaptation, information relating to the decrease and/or increase in the insulin dose, which depends on conditions that are dependent on the historical blood glucose level;

physical activity (sports), with entry of at least one weighting coefficient that is applicable to the insulin level to be calculated, in the event of physical activity.

For diabetes, the method includes a step of predicting the blood glucose level over time as a function of the type of physical activity, the expected duration, the historical blood glucose level, particularly during previous physical activity, and the planned nutrition, and optionally a step of forecasting caloric expenditure and a step of programming a glycemic alarm in the event of an overshoot of the individual's low and high threshold values.

Advantageously, the method comprises steps for entering data for the purpose of recording them, particularly for entering data relating to the protocol, and steps for displaying information, the entry and display being carried out by means of a remote entry, display, and communication interface system, such as a computer, a smartphone, or a tablet, which comprises a software application dedicated to the implementation of the method, the software application being preferably also capable of managing other parameters relating to the individual's physical activity, such as the individual's physical condition, their weight, tracing of the route by geolocation, their cardiac activity during exertion, their blood pressure, and their body temperature, and/or capable of connecting a community of individuals, each individual being able to make themself invisible or visible in relation to other individuals and being able to participate in private conversations, and/or capable of arranging information that is not specific to the individual relating to a chronic pathology (such as diabetes) and/or dietetics and/or physical activity, such as professional articles or specialized websites).

Preferably, the method comprises a step of secure identification of the individual implementing the method, and steps of identifying and/or inviting third parties accessing and/or receiving information relating to the method, particularly a step of inviting the individual's practitioner to record the data of the control protocol (adaptation protocol).

The method is such that it can include rapid access to certain information of the individual by reading a QR code.

The invention also relates to a computer program product that can be downloaded from a communication network and/or stored on a medium that can be read by computer, tablet, or smartphone and/or can be executed by a microprocessor and is characterized in that it comprises program code instructions for implementing the method of the invention.

### BRIEF DESCRIPTION OF THE DRAWINGS

The present invention will now be described with the aid of examples, which are only illustrative and in no way limit the scope of the invention, and on the basis of the accompanying illustrations.

FIG. 1 is a diagram of the device for implementing the method according to the invention for monitoring a physiological parameter of an individual.

FIGS. 2 and 3 are screenshots of the software application associated with the method and showing the conditions for reducing and respectively increasing the insulin dosage protocol.

FIGS. 4 and 5 illustrate screenshots of the software application showing the entry of information relating to the insulin dosing protocol and relating to physical activity.

### DETAILED DESCRIPTION OF THE PREFERRED EMBODIMENTS

The inventive method for controlling a physiological parameter of an individual will be exemplified here in relation to the chronic pathology of diabetes, the parameter to be monitored being the blood glucose level, so that the individual can regulate this level through administration of appropriate doses of insulin.

According to the invention, the method for managing or assisting in controlling or regulating the individual's blood glucose level comprises the steps of:

collecting the blood glucose level at a given time (called new blood glucose level) and recording the value for the blood glucose level in a database,

recording in the database of values collected from the blood glucose level at different moments in time in order to constitute a history of blood glucose level values, each of the values being associated with a precise moment, such as a time slot,

recording in the database of data relating to the individual's nutrition and/or physical activity, and preferably other parameters relating to the weather, temperature, and (geo)location, particularly in order to take the atmospheric pressure and the individual's state of mind/mood into account,

recording of information relating to the individualized protocol for adapting the insulin doses to be received by the individual,

processing of the information using electronic processing means and a computer program in order to transform them into a mathematical calculation algorithm capable of handling the automated calculation of the insulin dose,

automated calculation of at least one insulin dose influencing the blood glucose level following the recording of the new blood glucose level at a given time.

The automated calculation of an insulin dose at any given time takes the following into account:

history of blood glucose levels,

the given moment (the time slot) of the recording of the new blood glucose level, without necessarily having to take this new blood glucose level into account in the calculation,

the information items from the individualized protocol,

a low threshold value for the blood glucose level,

a high threshold value for the blood glucose level,

a carbohydrate level based on recorded nutrition-related data,

a weighting coefficient based on recorded data relating to physical activity and in the event of recorded physical activity.

The method allows—based on the insulin administration protocol established by the doctor specifically for the individual, and on the history of blood glucose levels, nutrition, physical activity, and on other parameters such as weather, temperature, (geo)location, atmospheric pressure (altitude), and the individual's state of mind—for the automatic calculation of the dose of ad hoc insulin that the individual must administer in real time.

Therefore, the method for monitoring blood glucose level is not only related to the measurement as such of the blood glucose level, but takes into account the level of carbohydrate (nutrition), the individual's physical activity, their current external environment, and their mood state.

The control method also makes it possible to record in a database the history of blood glucose levels combined with nutrition and physical activity in order to deduce therefrom statistical data intended to be subsequently processed by practitioners for example to improve the individual's protocol or protocols for other individuals, or carry out studies on the evolution of blood glucose levels according to the type of individual, the type of physical activity, and the type of nutrition.

The purpose of the method is to provide an insulin dose result either in real time or in a time projection.

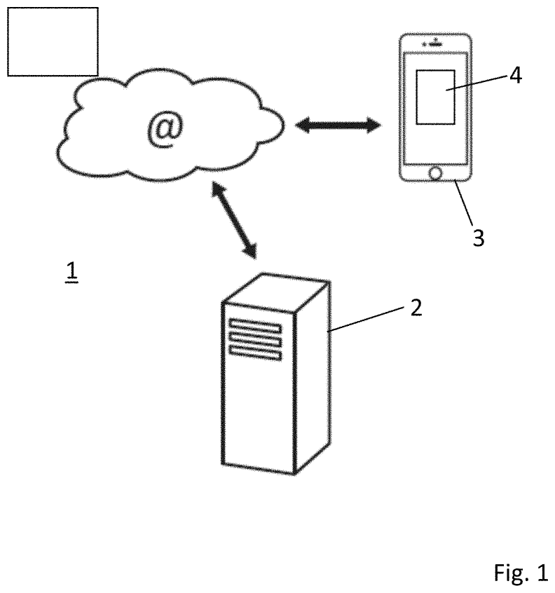

With reference to FIG. 1, the method of the invention is carried out by means of implementation means 1, which are at least a data storage and processing system 2 (hereinafter referred to as a data processing system), and an interface system 3 between the individual and the data processing system 2.

The interface system 3 comprises at least one display screen, means of remote communication, particularly via the internet and/or mobile communication networks, and an interface software application 4 that is dedicated to the method. Such an interface system 3 is a computer, a tablet, or a smartphone, for example, in which the dedicated software application 4 has been installed.

The data processing system 2 comprises at least remote communication means, data recording means, at least one database and data processing means comprising one or more microprocessors, and at least one computer program. The data processing system 2 is preferably hosted by a server-type hosting system.

The data processing system 2 is intended to receive the data transmitted by the interface system 3 and to process them in real time in order to deliver at least one evaluation result for an insulin dose when the dedicated software application interface 4 is implemented by recording a new blood glucose level.

All data entered/saved in the software application 4 via the interface system 3 are transmitted to the data processing system 2.

The means for implementing the evaluation method of the invention are designed so that each individual is identified when connecting to the interface software application 4 and by the data processing system 2.

Once the blood glucose level has been measured, the result of the assessment of the insulin dose, and/or other information related to the result, such as information on the condition of an individual who is in hypo- or hyperglycemia, for example, are to be recorded by the data processing system 2 and communicated to the individual, and optionally to one or more third parties. The information transmitted to third parties is selected according to the nature of the third party, for example as a function of whether the third party is a caregiver (relative, family of the individual) or a person supervising the individual/patient (doctor, pharmacist, nurse, etc.)

According to the invention, the control method further comprises an alert notification to the user/individual and the sending of alerts to third parties, either in an automated manner and/or on a voluntary basis by the user as a function of:

the result of the blood glucose level, particularly in the event of hypo- or hyperglycemia,

when the administered and recorded insulin dose does not match the calculated insulin dose, and/or

a lack of implementation of the control process.

The alert notification to the individual is performed audibly and/or visually via or on the interface system 3.

The sending of alerts, particularly in the event of hypo- or hyperglycemia, is performed automatically from the data processing system 2 or voluntarily by the individual from their software application via the interface system 3.

Preferably, the software application has an alert field that appears, for example, on the screen of the interface system 3 in the form of a single touch button. When the user presses the button, an alert information message is automatically transmitted to third parties registered for sending this type of alert. The alert information message transmitted to the third party is adapted to the reception means of the third party; it can be a telephone message, an e-mail, or a text message, for example.

Advantageously, the means for implementing the method further comprise a portable device for rapid identification of the patient and information relating to this patient, so that any third party can identify the individual and some of their vital information if the individual is unable to speak. In particular, the portable device comprises a QR Code. This portable device is in the form of a card or a bracelet, for example. It is not necessary for the third party to have the software application; scanning the QR Code enables access to information. The data processing system 2 comprises a database specific to the reading of the QR Code in order to access only a limited number of data items of the individual.

Regarding the information of the individualized protocol allowing the automated calculation of an insulin dose at a given moment/instant, the control method of the invention therefore comprises, prior to its daily implementation, a data recording step related to the protocol for adapting the insulin doses that the individual is intended to receive on a daily basis.

This protocol registration step is carried out by the referring practitioner, who must have previously downloaded the software application 4 on their interface system. The practitioner receives an invitation and authorization from the user/patient to enter, via the software application, several items of information in the database linked to the user's account. The information fields to be entered are offered step by step through data fields that are displayed during data entry. The items of information are the following:

type of diabetes;

type of device used to administer doses (pen or pump);

the unit of insulin dose used (mmol/L or mg/dL or g/L);

low and high threshold values for blood glucose levels;

the control periods, such as night, breakfast, morning, lunch, afternoon, dinner, evening, with a start time and an end time being assigned to each period;

meals when a meal is associated with a control period;

the insulin-related information for each period, especially the type of insulin (slow or fast), the name of the insulin, whether it is functional insulin therapy (yes or no), the dose range of insulin based on a blood glucose level value (multiple ranges can be entered associated with a blood glucose level value);

the adjustment of the insulin dose (adjustment or no adjustment) for a given period;

in the event of an adaptation, the selection of the adaptation method from among “the traditional method,” “the method by difference,” and “the difference method Δ”;

in the event of an adaptation, information relating to the decrease and/or increase in the insulin dose, which depends on conditions that are dependent on the historical blood glucose level;

physical activity (sports), with entry of at least one weighting coefficient applicable to the insulin level which will be calculated on the basis of the aforementioned information in the event of physical activity.

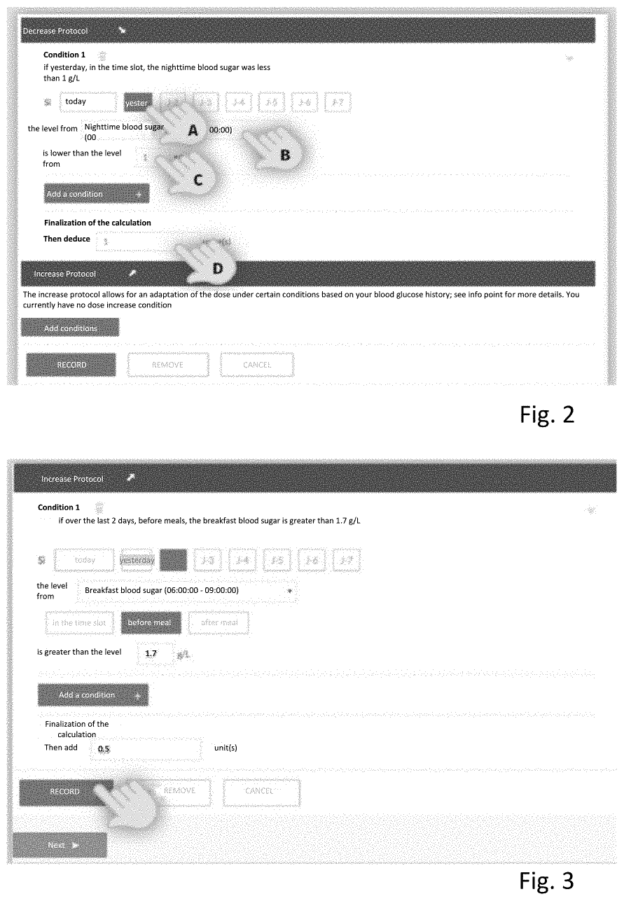

For information relating to the adaptation (decreasing and increasing) of the insulin dose, which is a function of conditions that are dependent on the historical blood glucose level, this includes the following data fields as shown in FIGS. 2 and 3, which show the decrease and increase conditions, respectively:

the date to choose from “today,” “yesterday,” “day-1,” “day-2” through “day-7,”

the period,

a threshold value for the blood glucose level associated with the period,

the unit decrease or increase value.

As an example, for a decrease condition (FIG. 2) that was associated with a given period such as breakfast, the various aforementioned fields make it possible to enter the information: “if yesterday the night blood glucose level was less than 1 g/L, then deduct 1 unit.”

As an example, for an increase condition (FIG. 3), the various aforementioned fields make it possible to enter the information: “if over the last 2 days, before a meal, the blood glucose level for breakfast was greater than 1.7 g/L, then add 0.5 units.”

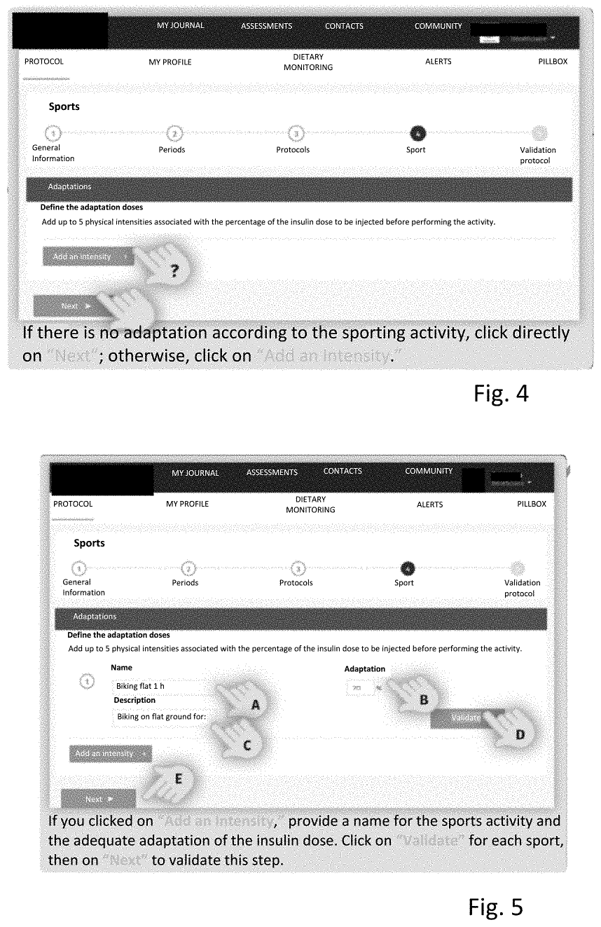

For information relating to the entry of physical activity in the protocol for a given period (FIGS. 4 and 5), it is preferably specified in combination with the weighting coefficient (for example 70%), the type of sport, and the duration (for example, cycling on flat ground for 1 hour). Thus, the calculation of the dose of insulin that is to be administered during the breakfast period, knowing that the individual will then be cycling for 1 hour, must in reality be reduced to 70% of the intermediate calculation made based on the previous protocol information.

Regarding the collection of the blood glucose level at a given time, it is done by measuring the blood glucose level and sending the measured level to the interface system 3.

The blood glucose level at a given time is measured by any appropriate means by sampling blood or interstitial fluid and means for analyzing the blood glucose level from the blood or from the interstitial fluid. There currently exists, for example, as an electronic measuring device without human intervention for the measurement, a patch that is stuck on the skin which, through the presence of a sensor, measures the blood glucose in the interstitial fluid and is suitable for transmitting the measurement information to a smartphone by scanning the sensor.

Once the measurement has been carried out, the measured value is entered manually into the interface system 3 or is transmitted thereto directly if the measuring device is a connected object.

All measurements of the blood glucose level over time that are entered into the interface system 3 are transmitted and recorded in the data processing system 2 in order to establish a database of the historical blood glucose level.

In addition, the blood glucose log and the blood glucose targets are recorded in the data processing system 2 via the interface system 3 and the software application 4.

The glycemic log includes not only the historical blood glucose level and the insulin doses administered, but also nutrition-related data on each meal, for instance, and hence the levels of carbohydrates as a function of nutrition, as well as the physical activity and the periods associated with these different parameters.

The glycemic objective is the thresholds for low and high blood glucose levels specific to each individual, which can be configured in the application and are to be recorded in the database. The low and high values correspond to the values that will trigger the alerts if the measured blood glucose level is below the low value (hypoglycemia) or above the high value (hyperglycemia).

Regarding the nutrition-related data, that is, the data relating to the foods and drinks consumed, these include data that are entered manually by the individual when they have exact knowledge of the level of carbohydrate associated with the food, the pre-recorded data corresponding to a list of foods that the user can select on the software application, the data being entered automatically by scanning and wireless remote connection such as Bluetooth, NFC, Wi-Fi. The nutrition-related data stored in the database is at least the name of the food, the carbohydrate level, and the associated glycemic index. Other data can be recorded in order to better identify foods, such as food family (e.g., mixed dishes, dairy products, condiments, drinks, fruits and vegetables, meats, fish, etc.), the type of food (for drinks, for example, wine, liquor and alcohol, juice, nectar, water, beer are listed), the brand, favorite foods, and in order to constitute a database that is useful to the practitioner, a non-exhaustive list of the ingredients of a food, the calories, the lipids, the proteins, the allergens, the calcium, the magnesium, the sodium, the potassium, the sulphate, the fluorine, the silica, the total content of mineral salts, practical food information, etc.

Regarding data relating to physical activity, these include data relating to the physical activity that the individual is about to perform (dose calculation planned for the activity performed afterward), or those just performed (preceding dose calculation). The data relating to physical activity include at least the type of physical activity, the duration planned, the recording of the blood glucose level just before the start of the activity, and optionally the entry of what is planned in terms of nutrition during activity, and recording of blood glucose level on arrival.

Depending on the type of physical activity, the expected duration, the historical blood glucose level in the same time slot or during a previous physical activity, and the planned nutrition, the data processing system 2 is able to carry out a forecast calculation of the blood glucose level over time and advantageously of the caloric expenditure, as well as the programming of a glycemic alarm in the event of an overshoot of the individual's low and high thresholds. When the actual blood glucose level measured just before the start of the activity is recorded, the forecast calculation is updated.

The type of physical activity is chosen, for example, from a menu listing different types of sport, such as running, walking, cycling, swimming, etc. It is possible to record an activity that was not listed beforehand in the application, and thus to record it in the database.

A distinction is made between data relating to recurrent physical activity that will have been recorded in the protocol data for the automated calculation of blood glucose doses, and data relating to physical activity that are recorded in real time and are not usual.

These latter data which are not included in the protocol are entered and saved in a data library outside of the data associated with the protocol. By associating these data relating to physical activity with the blood glucose level measurement data and recording them over time, it is possible, using the software application 4 or another software application, to compare these data with the calculations associated with the recorded personal protocol and to propose a new adaptation of the doses, and hence of the protocol.

The actual duration of physical activity is measured by a stopwatch that is integrated into the interface system 3.

Other parameters relating to physical activity can be recorded, including but not limited to weight, tracing of the route by geolocation, cardiac activity during exercise, blood pressure, body temperature, etc. All of these parameters are entered manually or supplied to the interface system 3 by connected means such as a fitness wristband or watch, blood pressure monitor, thermometer, or personal scale.

In addition to the data relating to nutrition and physical activity, the control method advantageously takes into account the parameters of the weather (e.g., temperature, humidity, atmospheric pressure), the temperature of the environment in which the individual is located when measuring the blood glucose level, their location, and the individual's state of mind/mood. The weather, temperature, and location are preferably recorded automatically by the geolocation of the interface system 3 and associated software applications, which provide information to the interface system. The state of mind is entered by the individual on the interface system by selecting one of the states proposed by the software application 4, for example from a scale of faces and/or a number scale of values and/or written expressions such as “happy,” “stressed,” “physical form,” etc.

The values of the aforementioned parameters (the individual's location, the weather, temperature, altitude, atmospheric pressure, humidity, and the individual's state of mind) are recorded in real time in data libraries and associated—e.g., for the physical activity and nutrition data recorded in real time—with the measured blood glucose levels so as to provide a data history in order to analyze them and study the behavior of the blood glucose in relation to these different parameters, making it possible to modify and adapt the individual's protocol. For example, an individual who lives by the sea and has a protocol that has been established accordingly in relation to their habits and goes on vacation at high altitude in the mountains, while possibly performing an activity that is different from the usual one, will observe a change in their blood glucose level. The study of the data history during their stay will make it possible to propose a protocol that is modified (and better adapted) compared to the usual one for the next time.

Therefore, the method for monitoring an individual's blood glucose level from the automated calculation of an insulin dose uses not only the measurement of the blood glucose level, but also data relating to nutrition and the individual's physical activity (in terms of their habits or at the time the measurements were taken), as well as data relating to the individualized insulin dosage protocol, with the software application dedicated to the process providing ease of data entry and, in particular, the individualized protocol. In order to obtain the most accurate calculation of the dose to be administered, additional parameters can be added, such as the individual's mood state, their geolocation, and the weather of the place where they are located.

Finally, the software application 4 is advantageously designed, not only for entering, transmitting, and displaying data concerning the individual, but also for:

accessing/consulting internet pages relating to diabetes, dietetics, and physical activity, particularly in order to consult professional articles providing information on diabetes and its environment;

interacting online (“chatting”) with a community of individuals using the same software application or in a private manner, the user being able to be visible or invisible in relation to other users, and if they make themself visible, they can be geolocated.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1-15. (canceled)

16. A method for managing or assisting in controlling or regulating a blood glucose level of an individual, comprising the following steps:
a preliminary step preceding a first implementation of the method, which comprises recording in a database information relating to an individual-specific protocol for adapting insulin doses, the recording being implemented by at least one dedicated software application and an entry and display interface system of the computer, tablet, or smartphone type;
collecting the blood glucose level at a given time and recording a value for the blood glucose level in the database, the collecting of the value for the blood glucose level being carried out by measuring the blood glucose level, and by recording the blood glucose level manually or orally, or even in an automated manner via a connected measurement system, on the entry and display interface system;
recording in the database of values collected from the blood glucose level at different moments in time to constitute a history of values of the individual's blood glucose level, each of the values being associated with a precise moment, such as a precise time slot;
optionally recording of data relating to at least one of nutrition or physical activity of the individual in the database via the entry and display interface system;
recording in the database of a location of the individual, the location being recorded automatically by a geolocation of the entry and display interface system used during the collection step;
processing of the recorded information using electronic processing means and a computer program to calculate in an automated manner at least one dose of insulin from the information from the individual-specific protocol, historical data on blood glucose levels, location, and optionally at least one of nutrition or physical activity.

17. The method according to claim 16, wherein, after the step of recording in the database of information relating to the individual-specific protocol for adapting insulin doses, and before a regular implementation of the method for managing by the individual, the method comprises a step of
processing the information of the protocol, using electronic processing means and a computer program, to transform the information of the protocol, in real time, into a mathematical calculation algorithm which will subsequently provide, also based on other data that will be recorded at least during the collection of the blood glucose level and the geolocation, for an automated calculation of the insulin doses at a given time for the individual.

18. The method according to claim 16, wherein the information of the adaptation protocol comprises personalized data relating to the individual's physical activity.

19. The method according to claim 16, wherein the step of recording in the database information relating to the individual-specific adaptation protocol is carried out only once before a regular implementation of the process by the user, the protocol being updatable by recording new associated information.

20. The method according to claim 16, wherein the automated calculation of an insulin dose at a given time is a function of
at least one of
at least one previous value for the blood glucose level,
a low threshold value for the blood glucose level, or
a high threshold value for the blood glucose level,
a weighting coefficient based on data relating to physical activity, and
a carbohydrate level based on nutrition-related data.

21. The method according to claim 16, further comprising
a step of recording and processing additional data during the collection of a value for a quantity influencing a physiological parameter, these additional data comprising at least one of
weather,
temperature,
altitude,
atmospheric pressure,
humidity,
a state of mind of the individual.

22. The method according to claim 16, further comprising at least one of
at least one step of alert notification to the user or individual, or
sending of at least one alert to a third party, at least one of automatically or voluntarily by the individual,
the alert being generated as a function of a result of at least one of
a value for a quantity influencing a physiological parameter,
a lack of implementation of a control method,
when a quantity influencing the physiological parameter and recorded during an additional recording step does not correspond to the quantity calculated.

23. The method according to claim 16, wherein the preliminary step of recording the information of the adaptation protocol is carried out by a practitioner of the individual.

24. The method according to claim 17, wherein the information of the protocol for adapting insulin doses comprises at least one of:
a type of diabetes;
a type of device used to administer the doses;
a unit of insulin dose used;
low and high threshold values for blood glucose levels;
control periods, including at least one of night, breakfast, morning, lunch, afternoon, dinner, or evening, with a start time and an end time being assigned to each period;
meals when a meal is associated with a control period;
insulin-related information for each period, including at least one of the type of insulin, a name of the insulin, whether the insulin is a functional insulin therapy, and a range of insulin dose according to a level value for blood glucose;
an adjustment of the insulin dose for a given period;
a selection of the adaptation method from among the methods offered on the entry and display interface system;
information relating to at least one of a decrease or increase in the insulin dose, which depends on conditions that are dependent on a historical blood glucose level; or
physical activity, with entry of at least one weighting coefficient that is applicable to the insulin level to be calculated, upon occurrence of physical activity.

25. The method according to claim 16, further comprising:
a step of predicting the blood glucose level over time as a function of a type of physical activity, an expected duration, a historical blood glucose level during previous physical activity, and a planned nutrition,
optionally a step of forecasting caloric expenditure, and
a step of programming a glycemic alarm upon an overshoot of the individual's low and high threshold values.

26. The method according to claim 16, wherein the software application being used to record the individual-specific adaptation protocol is also capable of managing other parameters associated with the physical activity of the individual, including at least one of
a physical condition of the individual,
a weight of the individual,
tracing of a route of the individual by geolocation,
cardiac activity of the individual during exertion,
blood pressure of the individual, and
body temperature of the individual.

27. The method according to claim 16, wherein the software application being used to record the individual-specific adaptation protocol is also configured to bring together a community of individuals, each individual being able to make themself invisible or visible in relation to other individuals and to at least one of
participate in private conversation, or
provide information that is not specific to the individual while relating to at least one of
a chronic pathology,
a diet, or
a physical activity,
to at least one of professional articles or specialized websites.

28. The method according to claim 16, further comprising steps of secure identification of the individual implementing said method, and
at least one of
identifying or inviting third parties who are at least one of accessing or receiving information relating to the method,
inviting a practitioner of the individual to record the data of the adaptation protocol.

29. The method according to claim 16, further comprising providing rapid access to certain information of the individual by reading a QR code.

30. A computer program product at least one of downloadable from a communication network or stored on a medium that can be at least one of read by computer, tablet, or smartphone, or executed by a microprocessor, comprising program code instructions to implement the method according to claim 16.
